(19) United States
(12) Patent Application Publication
(10) Pub. No.: US 2008/0095831 A1
(43) Pub. Date: Apr. 24, 2008

(54) TOPICAL FORMULATION OF MULTILAMELLAR VESICLES COMPOSITION FOR PERCUTANEOUS ABSORPTION OF PHARMACEUTICALLY ACTIVE AGENT

(76) Inventor: Thomas L. Mc Graw, Pompano Beach, FL (US)

Correspondence Address: FLEIT, KAIN, GIBBONS, GUTMAN, BONGINI;& BIANCO P.L., ONE BOCA COMMERCE CENTER, 551 NORTHWEST 77TH STREET, SUITE 111, BOCA RATON, FL, 33487, US

(21) Appl. No.: 11/463,646

(22) Filed: Aug. 10, 2006

(51) Int. Cl. A61K 9/127; A61K 31/192

(52) U.S. Cl. 424/450; 514/570

(57) ABSTRACT

A composition of multilamellar vesicles for the delivery of pharmaceutically-active substances through the skin of a mammal, the composition including between about 1 to about 30% Cyclomethicone 5-.N.F., between about 1 to about 30% PEG-12 Dimethicone; between about 3 to about 14% Cyclopentasiloxane and PEG 12 Dimethicone Crosspolymers, between about 0.9 to about 9% Lauryl PEG/PPG-18/18 Methicone, between about 2 to 5% Sepigel 305, between about 1 to about 4%, Carbopol ETD 2020 solution (2%), up to 100% Water, and Sodium hydroxide to a pH between about 6-6.5.

### BACKGROUND OF THE INVENTION

1. Field of the Invention

The present invention relates generally to a composition of multilamellar vesicles formulation useful in the percutaneous delivery of pharmaceutical active agents and more particularly relates to the composition of a formula that allows the incorporation of hydrophilic and hydrophobic drugs into silicone vesicles, thereby increasing the percutaneous absorption of the drugs.

2. Description of the Related Art

For some time now efforts have been made to develop an alternative to the well-known, and often unpleasant, oral and intravenous methods of introducing pharmaceuticals into the body. One quickly-developing area that shows great promise is the field of pharmaceutical penetration enhancers. Penetration enhancers are substances that facilitate absorption of pharmaceutical agents directly through the skin without the need for needles.

Two classes of penetration enhancers are solvents and amphiphiles. Examples of solvents enhancers are DMSO, methanol, or ethanol. Examples of amphiphiles are L and alpha amino acids, anionic or cationic surfactants, and phospholipids. Each of these classes has a tendency to cause a decrease in resistance through hydration or disruption of the stratum corneum. However, solvents suffer from the disadvantage of having a high potential for skin irritation and toxicity. For instance, DMSO currently has a limited utility because of its potential to cause ocular and dermal toxicity, as well as having an unpleasant taste and odor.

The art of microemulsion uses lipids or phospholipids (e.g., Phosphtadylcholine) in conjunction with other additives to topically enhance the delivery of a drug. Unfortunately, these lipids generally tend to leave a tacky or sticky residue on the skin. The uncomfortable feeling has been found to result in patient avoidance and noncompliance with drug regiments that are applied to skin.

Therefore a need exists to overcome the problems with the prior art as discussed above.

### SUMMARY OF THE INVENTION

Briefly, in accordance with one embodiment of the present invention, disclosed is a composition of multilamellar vesicles for the delivery of pharmaceutically-active substances through the skin of a mammal. One embodiment of the inventive composition includes Cyclomethicone 5-NF between about 1% to about 30% of the composition by weight, Peg-12 Dimethicone between about 1% to about 30% of the composition by weight, Cyclopentasiloxane and Peg-12 Dimethicone Crosspolymer between about 4% to about 14% of the composition by weight, Lauryl Peg/Ppg-18/18 Methicone between about 0.1% to about 9% of the composition by weight, Sepigel 305 between about 2% to about 5% of the composition by weight, carbopol ETD 2020 solution (2%) 2020 between about 1% to about 4% of the composition by weight, water between about 40% to about 100% of the composition by weight; and Sodium Hydroxide to obtain a pH of between about 6 to about 6.5. The Peg-12 Dimethicone forms vesicles, which are structurally comparable to liposomes and the Cyclomethicone 5-NF is used to improve the aesthetics of the composition. Several of the benefits are non-greasy, soft silky feel, excellent spreading, no oily build-up, non-stinging and reduces tackiness.

In accordance with an added feature of the invention, the composition includes a therapeutically effective amount of a pharmaceutically active substance and wherein the composition has a pH between about 6 to about 6.5. The pharmaceutically active substance can be, but is not limited to at least one of an antiemetic, such as Scopolamine, a prostaglandin, such as misoprostrol, an anti-inflammatory, such as ibuprofen, ketoprofen or diclofenac, a biologically active protein, such as phytospingosine or spingosine, and analgesic, such as ketamine and amitriptyline, a hormone, such as progesterone or testosterone, a steroid, such as clobesterol, a vasodilator, such as isosorbide dinitrate, a selective estrogen modulator, such as tamoxifen citrate, or other pharmaceutical agents.

Embodiments of the present invention also include a method of making a composition for percutaneous delivery of a pharmaceutically-active substance, where, if the active drug is water soluble, the method includes the following steps: dissolving the active drug in water, the dissolved active drug and the water together making up a Phase known as B1 and then agitating the Phase B1. Siloxane emulsifiers, which make up Phase A1, are incorporated into the Phase B1 while agitating so that the Phase A1 and Phase B1 form vesicles. Phase A1 and Phase B1 continue to be agitated while a sepigel solution 305 and a Carbopol ETD 2020 (2%) solution are added to the mixed Phases A1 and B1 to create an emulsion, the sepigel solution 305 and Carbopol ETD 2020 (2%) solution make up a Phase C. Finally, a sufficient quantity of sodium hydroxide is added to the mixture of Phases A1, B 1, and C to adjust a pH to between about 6 to about 6.5. However, if the active drug is not water soluble, the following steps take place, according to embodiments of the present invention. The active drug is incorporated in silicone emulsifers to form a smooth emulsion, the smooth emulsion is considered Phase A2. Phase A2 is mixed with water, the water being Phase B2, while agitating to form vesicles. The mixed Phases A2 and B2 are agitated while a sepigel solution 305 and a Carbopol ETD 2020 (2%) solution are added to the mixture of Phases A2 and B2, the sepigel solution 305 and Carbopol ETD 2020 (2%) solution being a Phase C, and a sufficient quantity of sodium hydroxide is added to the mixture of Phases A1, B1, and C to adjust a pH to between about 6 to about 6.5.

### DETAILED DESCRIPTION

While the specification concludes with claims defining the features of the invention that are regarded as novel, it is believed that the invention will be better understood from a consideration of the following description. It is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting; but rather, to provide an understandable description of the invention.

The present invention, according to one embodiment, overcomes problems with the prior art by providing multilamellar silicone vesicles in a composition that is rapidly absorbed through the skin so that active drugs within the composition are able to provide benefits, such as local treatment of wounds, scars, relief from pain, psoriasis, inflammation of skin, or other pathological conditions. Advantageously, the composition, in accordance with embodiments of the present invention, provides higher bioavailability of pharmaceutically active agents, thus providing an efficient vehicle for drug delivery in topical formulations.

As will be explained in detail below, a potential use for the Cyclomethicone 5-NF is as an excipient in the pharmaceutical topical formulation. The benefits encompass excellent spreading, a soft silky feel to the skin, and no oily residue or build-up. In addition, the inventive composition contains a detackifier and is non-cooling and non-stinging to the skin. Furthermore, the appearance is a colorless liquid with specific gravity at (77° F.) of 0.95, viscosity at 4.0 mm²·s⁻¹, and surface tension at 18.0 nm/m. It may be used alone or with other silicones or organic excipients.

One embodiment of the present invention includes a composition of Cyclomethicone 5-NF, Peg-12 Dimethicone, Cyclopentasiloxane (and) Peg-12 Dimethicone Crosspolymer, Lauryl Peg/Ppg-18/18 Methicone, Sepigel 305, Carbopol ETD 2020 (2%) solution, and water with a pH between about 6-6.5.

In other embodiments, the composition includes silicone wax (Stearoxytrimethylsilane and Stearyl Alcohol) and preservatives. By varying the percentages of silicone wax concentration, the volatility can be altered to vary the residence time of the silicone on the skin once the inventive composition is applied. The range of concentration of volatile siloxane is between about 1 to about 30%. In one embodiment of the invention, the composition is formulated with Cyclomethicone 5-NF (16%), Peg-12 Dimethicone (4%), Cyclopentasiloxane & Peg-12 Dimethicone (4%), Lauryl Peg/Ppg-18/18 Methicone (0.5%), Sepigel 305 (2%), Carbopol ETD 2020-(2%) solution (3%), water to (100%) and the pharmaceutical active substance to be delivered is Tamoxifen citrate (2%). The pH is adjusted to between about 6 to 6.5. This formulation has been found to improve the appearance of fibrous tissue and the redness of keloid scars. In one study about the potential use of tamoxifen citrate to treat keloids, Tamoxifen citrate in a concentration of about 0.01% was shown to have a deadly effect on keloid cells. The results of the study demonstrated Tamoxifen citrate's ability to inhibit keloid fibroblast production and decrease collagen production.

The emulsifier Peg-12 Dimethicone is a polyether functional siloxane. This siloxane is soluble in ethanol, is water dispersible, and provides interfacial surface tension reduction. This low viscosity liquid has a specific gravity of 1.03 and a viscosity of 360 cST. The benefits of this emulsifier are its ability to emulsify a variety of oils, and it ability to form silicone vesicles, which advantageously combines the aesthetic benefits with effective delivery. This emulsifier also provides a way to prepare silicone vesicles from a concentrated microemulsion by dilution of the emulsion into water to generate silicone vesicles. The concentration level of this emulsifier is from about 1 to about 30%. The vesicles can be loaded with active pharmaceuticals, such as Isosorbide Dinitrate, Ketoprofen, Ketamine, Amitriptline, Ibuprofen, Tamoxifen citrate, and other active ingredients. The unique nature of these vesicles allow both hydrophilic and hydrophobic active therapeutic agents to be incorporated.

Silicone vesicles are microstructures formed by adding emulsifier (Peg-12-Dimethicone) to water. These vesicles may be considered analogs to liposomes. There is currently a pharmaceutical need to incorporate lipophilic pharmaceutical agents into an aqueous finished product. This need can be met by encapsulating the water-incompatible actives by using amphiphilic colloidal system in microemulsions. The ability of Peg-12 Dimethicone to form vesicles creates a surfactant system wherein the surfactants rearrange themselves in a bilayer structure (lamellae). The hydrophobic moiety of the surfactant is oriented toward the inside of the bilayer while the hydrophilic moiety is facing the outside of the bilayer. Thereby, the hydrophilic and hydrophobic pharmaceutical active agents can be separated and protected from each other. The hydrophobic active agent can be distributed inside the bilayer while the hydrophilic pharmaceutically active agent will be in the solution inside the bilayer. This causes any pharmaceutically active agent, which might be irritating to the skin, to incorporate in the vesicle to prevent this irritation. These vesicles that are formed are very stable in an aqueous medium in concentrations of about 1 to about 30%. The flexibility of formulating with silicone emulsifier provides enhanced topical penetration, delivery of hydrophilic or hydrophobic drugs, and improved aesthetics. These silicone polymers provide a number of unique attributes, such as improved ease of spreadability, film formation, and reduced tackiness before and after absorption, leaving a smooth matte like finish.

Embodiments of the present invention provide the advantage that actives may be loaded in the same vesicle, with one as a water soluble phase and one in a silicone emulsifier phase. In one embodiment of the invention, the composition is formulated with Cyclomethicone 5-NF (16%), Peg-12 Dimethicone (4%), Cyclopentasiloxane & Peg-12 Dimethicone (4%), Lauryl Peg/Ppg-18/18 Methicone (0.5%), Sepigel 305 (2%), Carbopol ETD 2020-(2%) solution (3%), water to (100%) and the pharmaceutical active (2%) Ketamime and (2%) Amitriptyline. Then, pH is adjusted to between about 6 to about 6.5. Such formulation rapidly absorbs through the skin, which, for instance, provides relieve of pain and burning associated with postherpetic neuralgia. In another embodiment of the invention, the composition is formulated with Cyclomethicone 5-NF (16%), Peg-12 Dimethicone (4%), Cyclopentasiloxane & Peg-12 Dimethicone (4%), Lauryl Peg/Ppg-18/18 Methicone (0.5%), Sepigel 305 (2%), Carbopol ETD 2020-2% solution (3%) water to (100%) and the pharmaceutical active substance, progesterone (10%). pH is adjusted to between about 6 to about 6.5. Such formulation rapidly absorbs through the skin, where it provides relief from, for instance, postmenopausal symptoms.

In one embodiment of the present invention, the composition is formulated with prostaglandin (misoprostol). The composition is formulated with Cyclomethicone 5-NF (16%), Peg-12 Dimethicone (4%), Cyclopentasiloxane & Peg-12 Dimethicone (4%), Lauryl Peg/Ppg-18/18 Methicone (0.5%), Sepigel 305 (2%), Carbopol ETD 2020-2% solution (3%) and water to (100%), the pharmaceutically active substance Misoprostol (0.024%) and silicone wax (2% Stearoxytrimethylsilane and Stearyl Alcohol). pH is adjusted to between about 6 to about 6.5. This composition proved effective when used on diabetic foot ulcer. The healing time was shortened by the use of this composition.

The addition of silicone wax (2% Stearoxytrimethylsilane and Stearyl Alcohol) to the muiltillamellar vesicle formulation provides high substantivity to the active agents, as well as forming a protective film barrier on the surface of the skin. For example, a pressure ulcer is an area of skin and tissue that become broken down or injured. Embodiments of the present invention that include silicone wax have been proven to reduce the healing time of a pressure ulcer.

As an example, an experiment was conducted using a silicone wax (3%) and topical volatile silicone (Hexamethyldisiloxane, 97%) with ketoprofen. Testing was conducted on the forearm of five panelists. Semi-quantitative analysis of ketoprofen remaining on the skin of the panelist was analyzed by a spectascope. The experiment showed the substantivity of the silicone gum helped increase substantivity of ketoprofen. After 40 minutes, only traces of ketoprofen was detected on the skin exposed to formulation without the silicone gum, whereas after 6 hours, ketoprofen was still detected from the formulation containing silicone gum. The film formed after application helped maintain active agents in contact with the skin by creating a reservoir for the agents.

Another experiment was conducted to determine penetration of actives through hairless rat skin. The experiment was carried out in a Franz cell at 32° C. with NACL Receptors medium. The silicone formulation was composed of Hexamethyldisiloxane, silicone gums and the actives (hydrocortisone (0.05%) or ibuprofen (5%)). These silicone formulations were compared to commercial products without silicone. The silicone formulation showed a 235% increase in the penetration rate of ibuprofen over the standard commercial product. In addition, there was a 35-fold increase of the active (hydrocortisone) within the stratum corneum, when compared to the standard formulation.

Lauryl Peg/Ppg 18/18 Methicone tends to make a very stable emulsion when used as a co-emulsifier in a topical formula. A few of the benefits of this emulsifier are ease of spreading, resistance to washing off, lack of greasy feel, and protection. The percentage of concentration is between about (0.5%-9%).

A few exemplary benefits of silicone elastomers blend (Cyclopentasiloxane (and) PEG-12 Dimethicone Crosspolymer), are powdery after feel, reduced tack, and a stable emulsion. This elastomer creates high water content and low to high viscosity. The use level of this elastomer is 4-14%. The specifications for these elastomers are: 0.96 specific gravity, 12.5% nonvolatile content, and viscosity at (77° F.)<100 cp. In accordance with the present invention, water is prepared in silicone emulsion to provide improved aesthetics. In accordance with other features of the present invention, crosspolymer concentration is between about 4-14%. This material can bring a wide range of aesthetic, pharmacokinetic to topical pharmaceuticals. The silicone exicipients provide an ideal environment for improving drug diffusion and release ratios.

In one embodiment of the invention, the composition is formulated with Cyclomethicone 5-NF (16%), Peg-12 Dimethicone (4%), Cyclopentasiloxane & Peg-12 Dimethicone (4%), Lauryl Peg/Ppg-18/18 Methicone (0.5%), Sepigel 305 (2%), Carbopol ETD 2020-2% solution (3%) and water to (100%) and the pharmaceutically active substance to be delivered is nicotinamide (4%), with the pH adjusted to between 6 to 6.5. In tests, such formulation rapidly absorbed through the skin. This composition was compared to a standard clindamycin 1% solution and proved to be clinically equivalent when treating acne vulgaris.

Several pharmacokinetic benefits of the silicone excipients are substantivity, rub off resistance, increased bioavailability, and managed release. In one embodiment of the topical muiltilamellar vesicles formulations, the addition of misoprolol and silicone wax (3%) increased the substantivity on the skin due to film-forming properties. In another aspect of the invention, the composition is formulated with a non-steroid anti-inflammatory surfactant agent, diclofenac, such formulation is rapidly absorbed and provides local pain relief.

The Carbopol ETD 2020 polymer is specifically designed for thickening the surfactant system. It is a cross-linked polyacrylic acid copolymer processed in a toxicologically preferred cosolvent system, which delivers excellent thickening and suspending capabilities.

In other embodiments, Sepigel solution 305 is used at 2% concentration to help emulsify all types of oil phases without heating, which produces a gel-cream with a rich silky texture. In one embodiment of this invention, the multillamellar vesicle composition contains isorbide dinitarate in 0.2% concentration. In an experiment, this concentration was applied to the skin of a diabetic patient, which had diabetic neuropathy. Over a 4-week period, treatment resulted in improved reduction in pain and burning for the patient.

Stearoxytrimethylsilane and stearyl alcohol (silicone wax) is an excipient known to those of skill in the art and typically used in pharmaceutical applications as a thickening agent and water repellent. In accordance with embodiments of the present invention, this wax is added to the compositions described above to slow absorption of the active agents and allow the composition to remain in contact with the skin for an extended period, wherein it will form a semi-occlusive film on the surface.

Compositions, in accordance with the present invention, are applied topically as frequently as required to achieve the desired therapeutic response. In other words, the compositions are applied in sufficient amounts to provide the desired effect without undesirable side effects of the drug.

In one embodiment of the invention Clobesterol propionate (0.025%) was incorporated into multilamellar vesicle formulation and was then compared to the standard pharmaceutical product of Clobesterol (0.05%) ointment. Both products improved plaque psoriasis patches on the elbow. The multilamellar vesicle composition provided the same therapeutic response with a lower concentration of the active pharmaceutically agent. This composition, due to its vesicle formation, allows the controlled release of the active and improves the bioavailability of the active by affecting the penetration of the active through the skin. The standard pharmaceutical product caused less patient compliance due to very poor aesthetics. Patients were less likely to apply the standard regiment because of greasiness and transference of the ointment to their clothes. In contrast, the multilamellar vesicle composition of the present invention was quickly absorbed, leaving a non-greasy and non-transferable substantive film on the surface. A wide range of studies document the low order of toxicity of the silicone additives in the inventive composition. Advantageously, none of the ingredients are toxic to the skin, nor are the ingredients irritating or sensitizing.

The following nine examples are provided for purpose of illustration, but not of limitation.

### EXAMPLE #1

The pharmaceutical active agent, tamoxifen citrate 0.1% is incorporated into a multilamellar vesicle composition. This composition can be used to improve the appearance of keloid scars.

The following describes the preparation of Tamoxifen citrate 0.1% (100 mg) in multilamellar vesicles composition. Tamoxifen is a triphenyletylene derivative, non steroidal antiestrogen. Tamoxifen citrate is freely soluble in 0.5 mg/ml in water at pka 8.85. The composition is formulated to 100 gms of final product.

Phases:

| | Wt. % |
|---|---|
| Phase A - Silicone Phase | |
| Cyclomethicone 5-NF | 16% |
| Peg-12 Dimethicone | 4% |
| Cyclopentasiloxane and Peg-12 Dimethicone crosspolymer | 4% |
| Lauryl Peg/Ppg-18/18 Methicone | 0.5% |
| *Stearoxytrimethylsilane | 2% |
| Phase B - Aqueous Phase | |
| Tamoxifen citrate | 100 mg |
| Water | to 100% |
| Phase C | |
| Sepigel Solution 305 | 2% |
| Carbopol ETD 2020 (2%) solution | 3% |
| Phase D | |
| Sodium Hydroxide to a pH of about 6 to 6.5 | qs |

This formulation is released at a slower rate due to the sustainability of the siloxane wax (Stearoxytrimethylsilane and stearyl aclcohol) and its resulting ability to remain in active contact with the skin over a longer period of time.

Formulation:
1. Dissolve the active agent, which is a preparation of tamoxifen citrate 0.1%, in the aqueous phase, Phase (B), and mix well;
2. Mix all Phase (A) ingredients together;
3. Add Phase (A) ingredients to Phase (B) ingredients, then agitate to form vesicles;
4. Combine ingredients in Phase (C), then add Phase (C) to Phase (A+B) emulsion;
5. Homogenize Mixture;
6. Add enough Phase D to the emulsion to achieve a pH between about 6 to about 6.5

### EXAMPLE #2

The pharmaceutical active agent, isosorbide dinitrate 0.2%, is incorporated into a multilamellar vesicle composition. This composition can be used to treat chronic painful diabetic neuropathy.

The following describes the preparation of isosorbide dinitrate 0.2% in a multilamellar vesicles composition. Isosorbide dinitrate is an organic nitrate which is a vasodilator. It occurs as crystalline powder, which is sparingly soluble in water. The composition is formulated to 100 gms of final product.

Phases:

| | Wt % |
|---|---|
| Phase A - Silicone Phase | |
| Isosorbide Dinitrate | 0.2% |
| Cyclomethicone 5 NF | 16% |
| Peg-12 Dimethicone | 4% |
| Cyclopentasiloxane and Peg-12 Dimethicone Crosspolymer | 4% |
| Lauryl Peg/Ppg-18/18 Methicone | 0.5% |
| Phase B - Aqueous Phase | |
| Water | to 100% |
| Phase C | |
| Sepigel Solution 305 | 2% |
| Carbopol ETD 2020 (2%) solution | 3% |
| Phase D | |
| Sodium Hydroxide to a pH of about 6 to 6.5 | qs |

Formulation:
1. Incorporate the active, isosorbide dinitrate 0.2%, within silicone phase (Phase A) and mix to form a smooth emulsion;
2. Add phase (A) to Phase (B) and agitate to form vesicles; 3. Combine Phase (C) ingredients, then add Phase (C) to Phase (A+B) emulsion;
4. Homogenize Mixture; and
5. Add a sufficient amount of Sodium Hydroxide, Phase (D) to Phase (A+B+C) emulsion to achieve a pH level of about 6 to about 6.5.

### EXAMPLE #3

The pharmaceutically active agent of misoprostol 0.05% is incorporated into a multilamellar vesicle composition. This composition can be used to aid in the healing of wounds or ulcers.

The following describes the preparation of misoprostol 0.05% in a multilamellar vesicles composition. Misoprostol is a synthetic analog of prostaglandin E1 and is a water-soluble viscous liquid. In one embodiment of the present invention, the composition is formulated to 100 gms. of final product.

Phases:

| | Wt % |
|---|---|
| Phase A - Silicone Phase | |
| Cyclomethicone 5-NF | 16% |
| Peg-12 Dimethicone | 4% |
| Cyclopentasiloxane and Peg-12 Dimethicone crosspolymer | 4% |
| Lauryl Peg/Ppg-18/18 Methicone | 0.5% |
| Phase B - Aqueous Phase | |
| Misprostol | 0.05% |
| Water | to 100% |
| Phase C | |
| Sepigel Solution 305 | 2% |
| Carbopol ETD 2020 (2%) | 3% |
| Phase D | |
| Sodium Hydroxide to a pH of about 6 to 6.5 | qs |

Formulation:
1. Dissolve the active agent, which is a preparation of misoprostol 0.05%, in the aqueous phase, Phase (B), and mix well;
2. Mix all Phase (A) ingredients together;
3. Add Phase (A) ingredients to Phase (B) ingredients, then agitate to form vesicles;
4. Combine ingredients in Phase (C), then add Phase (C) to Phase (A+B) emulsion;
5. Homogenize mixture; and
6. Add enough Phase D to the emulsion to achieve a pH between about 6 to about 6.5.

### EXAMPLE #4

The pharmaceutical active agent of ketoprofen 5% is incorporated into a multilamellar vesicle composition. This composition can be used for the relief of localized inflammation or pain.

The following describes the preparation of ketoprofen 5% in a multilamellar vesicles composition. Ketoprofen is a non steroid anti-inflammatory occurring as a fine to granulated powder. The pka of ketoprofen is 5.9. Ketoprofen 5% is insoluble in water. This composition is formulated to 100 gms. of final product.

Phases:

| | Wt. % |
|---|---|
| Phase A - Silicone Phase | |
| Cyclomethicone 5 NF | 16% |
| Peg-12 Dimethicone | 4% |
| Cyclopentasiloxane and peg-12 Dimethicone Crosspolymer | 4% |
| Lauryl Peg/PPG-18/18 Methicone | 0.5% |
| Ketoprofen | 5% |
| Phase B - Aqueous Phase | |
| Water | 100% |
| Phase C | |
| Sepigel Solution 305 | 2% |
| Carbopol ETD 2020 (2%) | 3% |
| Phase D | |
| Sodium Hydroxide to a pH of about 6 to 6.5 | qs |

Formulation:
1. Incorporate the active, ketoprofen 5%, within silicone Phase (Phase A) and mix to form a smooth emulsion;
2. Add phase (A) to Phase (B) and agitate to form vesicles;
3. Combine Phase (C) ingredients, then add Phase (C) to Phase (A+B) emulsion;
4. Homogenize Mixture; and
5. Add sufficient amount of Sodium Hydroxide, Phase (D), to achieve a pH level of about 6 to about 6.5.

### EXAMPLE #5

The pharmaceutical active agents of of ketamine hydrochloride 2% and amitriptyline 2% are incorporated into a multilamellar vesicle composition. This composition is used for the relief of release pain associated with diabetic neuropathy.

The following describes preparation of ketamine hydrochloride 2% and amitriptyline 2% in a multilamellar vesicles composition as an anesthetic and analgesic agent. Ketamine is soluble in water (1 gm. in 4 mls.) Ketamine hydrochloride is equivalent to 1.15 mg of ketamine base. Amitriptline is a tricyclic antidepressant and is freely soluble in water with a 1 to 1 ratio. The composition is formulated to make 100 grams of the product. This composition is proven to release pain associated with diabetic neuropathy.

Phases:

| | Wt. % |
|---|---|
| Phase A - Silicone Phase | |
| Cyclomethicone 5-NF | 16% |
| Peg-12 Dimethicone | 4% |
| Cyclopentasiloxane and Peg-12 Dimethicone crosspolymer | 4% |
| Lauryl Peg/Ppg-18/18 Methicone | 0.5% |
| Phase B - Aqueous Phase | |
| Ketamine hydrochloride | 2% |
| Amitriptyline hydrochloride | 2% |
| Water | to 100% |
| Phase C | |
| Sepigel solution 305 | 2% |
| Carbopol ETD 2020 (2%) Solution | 3% |
| Phase D | |
| Sodium Hydroxide to a pH of about 6 to about 6.5 | qs |

Formulation:
1. Dissolve the active agent, which is ketamine hydrochloride 2% and amitriptyline 2%, in the aqueous phase, Phase (B) and mix well;
2. Mix all Phase (A) ingredients together;
3. Add Phase (A) ingredients to Phase (B) ingredients, then agitate to form vesicles;
4. Combine ingredients in Phase (C), then add Phase (C) to Phase (A+B) emulsion;
5. Homogenize Mixture; and
6. Add enough Phase D to the emulsion to achieve a pH between about 6 to about 6.5.

### EXAMPLE #6

The pharmaceutical active agent of clonidine hydrochloride 0.1% is incorporated into a multilamellar vesicle composition. This composition with clonidine 0.1% is often used to treat various types of pain.

The following describes preparation of clonidine hydrochloride 0.1% in a multilamellar vesicles composition. This composition is an imidazoline derivative generally used as a central alpha-2 adrenergic agent. It occurs as crystalline powder, which is freely soluble in water with a pH range of 3.5 to 5. The composition is formulated to about 100 gms. of final product.

Phases:

| | Wt. % |
|---|---|
| Phase A - Silicone Phase | |
| Cyclomethicone 5-NF | 16% |
| Peg-12 Dimethicone | 4% |
| Cyclopentasloxane and PEG-12 Dimethicone crosspolymer | 4% |
| Lauryl Peg/Ppg-18/18 Methicone | 0.5% |
| Phase B - Aqueous Phase | |
| Clonidine hydrochloride | 0.1% |
| Water | to 100% |
| Phase C | |
| Sepigel Solution 305 | 2% |
| Carbopol ETD 2020 (2%) solution | 3% |
| Phase D | |
| Sodium Hydroxide to a pH of about 6 to about 6.5 | qs |

Formulation:
1. Dissolve the active agent, which is clonidine hydrochloride 0.1%, in the aqueous phase, Phase (B), and mix well;
2. Mix all Phase (A) ingredients together;
3. Add Phase (A) ingredients to Phase (B) ingredients, then agitate to form vesicles;
4. Combine ingredients in Phase (C), then add Phase (C) to Phase (A+B) emulsion
5. Homogenize Mixture; and
6. Add enough Phase (D) to the emulsion to achieve a pH between about 6 to about 6.5.

### EXAMPLE #7

The pharmaceutical active agent of scopolamine hydrobromide 0.25% is incorporated into a multilamellar vesicle composition. This composition with scopolamine hydrobromide 0.25% is used to prevent nausea cause by motion sickness.

The following describes preparation of scopolamine hydrobromide 0.25% in a multilamellar vesicles compositions. Scopolamine hydrobromide is a naturally occurring tertiary amine anticholinergic also known as “hyoscine.” It is available as a hydrobromide salt in powder form. The drug is soluble to the extent of 0.67 gm/ml in water. It is formulated to 100 gms. of final product.

Phases:

| | Wt. % |
|---|---|
| Phase A - Oil Phase | |
| Cyclomethicone 5-NF | |
| 16% Peg-12 Dimethicone | 4% |
| Cyclopentasiloxane and PEG-12 Dimethicone crosspolymer | 4% |
| Lauryl PEG/PPG-18/18 Methicone | 0.5% |
| Scopolamine hydrobromide | 0.25% |
| Phase B - Aqueous Phase | |
| Water | to 100% |
| Phase C | |
| Sepigel solution 305 | 2% |
| Carbopol ETD 2020 (2%) | 3% |
| Phase D | |
| *pH Buffer 5.0 buffer | 0.5% |

Formulation:
1. Incorporate the active Scopolamine hydrobromide 0.25% within silicone phase (Phase A) and mix to form a smooth emulsion;
2. Add phase (A) to Phase (B) and agitate to form vesicles;
3. Combine Phase (C) ingredients, then add Phase (C) to Phase (A+B) emulsion;
4. Homogenize Mixture; and
5. Add Phase D together (*In Phase D, a pH buffer of 5.0 should to be prepared. This is accomplished by mixing 0.1 M citric acid in purified water with 0.2 M disodium phosphate in purified water in 1 to 1 ratio), then add 0.5% to final emulsion.

### EXAMPLE #8

The pharmaceutically active agent of progesterone 10% is incorporated into a multilamellar vesicle composition. This composition with progesterone 10% is often used to treat postmenopausal symptoms.

The following describes preparation of progesterone 10% in a multilamellar vesicles composition. Progesterone is a naturally occurring progestin that occurs as a crystalline powder that is insoluble in water. It melts and exists as a polymorph a 121 degrees Celsius. The composition is formulated to yield 100 gms of final product.

Phases:

| | Wt. % |
|---|---|
| Phase A - Oil Phase | |
| Cyclomethicone 5-NF | 16% |
| Peg-12 Dimethicone | 4% |
| Cyclopentasiloxane and Peg-12 Dimethicone crosspolymer | 4% |
| Lauryl PEG/PPG-18/18 Methicone | 0.5% |
| Progesterone micronized | 10% |
| Phase B - Aqueous Phase | |
| Water | to 100% |
| Phase C | |
| Sepigel Solution 305 | 2% |
| Carbopol ETD 2020 (2%) | 3% |
| Phase D | |
| Sodium Hydroxide to a pH of about 6 to 6.5 | qs |

Formulation:
1. Incorporate the active, progesterone 10%, within silicone phase (Phase A) and mix to form a smooth emulsion;
2. Add phase (A) to Phase (B) and agitate to form vesicles.
3. Combine Phase (C) ingredients, then add Phase (C) to Phase (A+B) emulsion.
4. Homogenize Mixture; and 5. Add sufficient amount of Sodium Hydroxide, Phase (D), to achieve a pH level of about 6 to about 6.5.

### EXAMPLE #9

The pharmaceutical active agent of nicotinamide 4% is incorporated into a multilamellar vesicles composition. This composition can be used for the treatment of acne vulgaris. This formulation is as effective as clindamycin for treatment of moderate inflammation of acne vulgaris.

The following describes preparation of nicotinamide 4% in a multilamellar vesicles composition, which is a water soluble b-complex vitamin that occurs as a crystalline powder. It is freely soluble in water and has a pka value of 0.5. Its solution is neutral to litmus. The nicotinamide is mixed 4 grams in 100 grams of vehicle.

Phases:

| | Wt. |
|---|---|
| Phase A - Oil Phase | |
| Cyclomethicone 5-NF | 16% |
| Peg-12 Dimethicone | 4% |
| Cyclopentasiloxane and Peg-12 Dimethicone crosspolymer | 4% |
| Lauryl PEG/PPG-18/18 Methicone | 0.5% |
| Phase B - Aqueous Phase | |
| Water | to 100% |
| Nicotinamide | 4% |
| Phase C | |
| Sepigel Solution 305 | 2% |
| Carbopol ETD 2020 | 3% |
| Phase D | |
| Sodium Hydroxide to a pH of about 6 t o7 | qs |

Formulation:
1. Dissolve the active agent, which is nicotinamide 4%, in the aqueous phase, Phase (B), and mix well;
2. Mix all Phase (A) ingredients together;
3. Add Phase (A) ingredients to Phase (B) ingredients, then agitate to form vesicles;
4. Combine ingredients in Phase (C), then add Phase (C) to Phase (A+B) emulsion;
5. Homogenize mixture; and
6. Add enough Phase D to the emulsion to achieve a pH of between about 6 to about 6.5.

The following is a composition of silicone in water emulsion according to embodiments of the present invention.

Ingredients:

Wt:

| | |
|---|---|
| Phase A - Oil Phase | |
| Cylomethicone 5 NF | 1–30% |
| Peg-12 Dimethicone | 1–30% |
| Cyclopentasilicone and Peg-12-Dimethicone Crosspolymer | 4–14% |
| Lauryl Peg/Ppg-18/18 Methicone | 0.1–9% |
| Phase B: Aqueous Phase | |
| Water | to 100% |
| Phase C: | |
| Sepigel 305 | 2 to 5% |
| Carbopol ETD 2020 (2%) solution | 1 to 4% |
| Phase D: | |
| Sodium hydroxide | qs |

Procedure:
1. Preparation of active:
   *If the active is water-soluble, the active is dissolved in water and the mixture becomes Phase (B). Phase (B) is then agitated.
   *If the active is water insoluble, the active is incorporated into the silicone Phase (A) and mixed to form a smooth emulsion.
2. Add Phase (A) slowly to Phase (B) with agitation;
3. Add Phase (C) to Phase (A+B) emulsion;
4. Homogenize the mixture; and
5. Add Phase (D) to Phase (A+B+C) in sufficient quantity to adjust pH to between about 6 to about 6.5.

The present invention, which relates to a composition useful in the percutaneous delivery of pharmaceutically active agents, has been described. Due to the nature of this multilamellar vesicles formation, drugs such as vasodilators, anti-inflammatory, prostaglandins, hormones, analgesics, antiemetics, and others are rapidly absorbed through the skin to provide local therapeutic concentration. The invention elicits a therapeutic response by penetrating into the epidermis and/or dermis. The use of this composition accomplishes improved product aesthetic and higher bioavailability of the pharmaceutical active agent, thus providing an efficient vehicle for drug delivery in topical formulations.

Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments, and it is intended that the appended claims cover any and all such applications, modifications, and embodiments within the scope of the present invention.

The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically.

What is claimed is:

1. A composition of multilamellar vesicles for the delivery of pharmaceutically-active substances through the skin of a mammal, the composition comprising:
cyclomethicone 5-NF between about 1% to about 30% of the composition by weight;
Peg-12 Dimethicone between about 1% to about 30% of the composition by weight;
cyclopentasiloxane and Peg-12 Dimethicone Crosspolymer between about 4% to about 14% of the composition by weight;
lauryl Peg/Ppg-18/18 Methicone between about 0.1% to about 9% of the composition by weight;
sepigel 305 between about 2% to about 5% of the composition by weight;
carbopol ETD 2020 solution (2%) 2020 between about 1% to about 4% of the composition by weight;
water between about 40% to about 100% of the composition by weight; and
sodium hydroxide to obtain a pH of between about 6 to about 6.5.

2. The composition according to claim 1, further comprising:
a therapeutically effective amount of a pharmaceutically active substance.

3. The composition according to claim 2, wherein the pharmaceutically active substance is at least one of:
an antiemetic;
a prostaglandin;
an anti-inflammatory;
a biologically active protein;
an analgesic;
a hormone;
a steroid;
a vasodilator; and
a selective estrogen modulator.

4. A composition according to claim 1, further comprising one of:
ketoprofen between about 1% to about 10%;
ibuprofen between about 1% to about 10%; and
diclofenac between about 1% to about 10%.

5. The composition according to claim 1, further comprising one of:
progesterone between about 1% to about 10%; and
testosterone between about 1% to about 10%.

6. The composition according to claim 1, further comprising:
misoprostol between about 0.024% to about 1%.

7. The composition according to claim 6, further comprising:
metronidazole between about 0.75% to about 5%.

8. The composition according to claim 1, further comprising:
scopolamine between about 0.75% to about 3%.

9. The composition according to claim 1, further comprising:
tamoxifen citrate between about 0.2% to about 5%.

10. The composition according to claim 1, further comprising:
isosorbide dinitrate between about 0.1% to about 2%.

11. The composition according to claim 1, further comprising:
clobestrol propionate between about 0.025% to about 2%.

12. The composition according to claim 1, further comprising:
ketamine between about 1% to about 10%.

13. The composition according to claim 12, further comprising:
amitriptyline between about 1% to about 10%.

14. The composition according to claim 1, further comprising one of:
phytospingosine between about 0.01% to about 5%; and
spingosine between about 0.01% to about 5%.

15. The composition according to claim 1, further comprising:
stearoxytrimethylsilane; and
stearyl alcohol.

16. A method of making a composition for percutaneous delivery of an active drug, the method comprising:
in response to the active drug being water soluble:
dissolving the active drug in water, the dissolved active drug and the water together being a Phase B1;
agitating the Phase B1;
incorporating siloxane emulsifiers (Phase A1), into the Phase B1 while agitating the Phase A1 and Phase B1 to form vesicles; and
agitating the Phase A1 and Phase B1 while:
adding a sepigel solution 305 and a Carbopol ETD 2020 (2%) solution, to the Phases A1 and B1 to create an emulsion, the sepigel solution 305 and Carbopol ETD 2020 (2%) solution being a Phase C; and
adding a sufficient quantity of sodium hydroxide to the Phases A1, B1, and C to adjust a pH to between about 6 to about 6.5; and
in response to the active drug not being water soluble:
incorporating the active drug in silicone emulsifers to form a smooth emulsion, the smooth emulsion being a Phase A2;
mixing the Phase A2 with water (Phase B2) while agitating to form vesicles; and
agitating the mixed Phases A2 and B2, while:
adding a sepigel solution 305 and a Carbopol ETD 2020 (2%) solution to the mixture of Phases A2 and B2, the sepigel solution 305 and Carbopol ETD 2020 (2%) solution being a Phase C; and
adding a sufficient quantity of sodium hydroxide to the mixture of Phases A1, B1, and C to adjust a pH to between about 6 to about 6.5.
